(19) United States
(12) Patent Application Publication
(10) Pub. No.: US 2018/0338797 A1
(43) Pub. Date: Nov. 29, 2018

(54) CATHETER TRACKING

(71) Applicant: SV-Medtech, Inc., Livermore, CA (US)

(72) Inventors: Thomas C. Moore, Livermore, CA (US); Kendall R. Waters, Sammamish, WA (US); Robert Zelenka, Milpitas, CA (US)

(73) Assignee: SV-Medtech, Inc., Livermore, CA (US)

(21) Appl. No.: 15/990,355

(22) Filed: May 25, 2018

Related U.S. Application Data
(60) Provisional application No. 62/512,142, filed on May 29, 2017.

(51) Int. Cl. A61B 34/20; A61B 5/06; A61B 5/00

(52) U.S. Cl. CPC A61B 34/20; A61B 5/065; A61B 2034/2055; A61B 2562/0223; A61B 2562/0233; A61B 5/742

(57) ABSTRACT

A catheter tracking system can be used to track motion and/or position of a catheter during delivery into a patient. A catheter tracking system can include an encoder and a guide roller to guide delivery of a catheter. The encoder can be configured to track motion or position the guide roller. The encoder may be a rotary optical encoder or a magnetic position sensor. A processing unit can also be used to determine motion or position of the catheter. The catheter tracking device may also include a wireless transmitter for communicating the catheter motion or position to an external device.

### CROSS-REFERENCE TO RELATED APPLICATIONS

The present application claims priority to U.S. Provisional Patent Application No. 62/512,142, filed on May 29, 2017, the disclosure of which is hereby incorporated by reference in its entirety.

### INTRODUCTION

Catheters are inserted into the human body to perform a broad range of functions, including use of intravascular catheters for imaging blood vessels. Knowing precise position information of the intravascular catheters can provide an operator valuable information regarding spatial variation of tissue structure, tissue composition, and physiological function.

Catheter accessories, such as catheter pullback devices, enable automated motion and position tracking of catheters. The catheter pullback devices are generally bulky, heavy, and require cabling to provide power. These characteristics can be problematic in clinical environments, particularly in a sterile field where use of a sterile drape or bag may be required to maintain a sterile barrier. Further, the use of such catheter accessories may disrupt common catheter-based clinical workflows wherein an operator may prefer to manually control catheter position.

FIG. 1 is a sectional view of a prior art hemostasis valve 10 that can be used by a physician to facilitate delivery of intravascular devices into the cardiovascular system of a patient. The hemostasis valve 10 may include a flush port 12, a valve 14, and a luer fitting 16. A guiding catheter (not shown) that is positioned within a patient's vascular system may be connected to the luer fitting 16. Saline may be delivered through the flush port lumen 18 to the hemostasis valve 10 and guiding catheter. Intravascular devices, such as intracoronary imaging catheters, can be delivered through an entry port 20, an exit port 22, and into guiding catheter in the vascular system of a patient. The valve 14 may have a Tuohy-Borst valve design that includes a seal to minimize blood loss during a diagnostic or interventional procedure.

### SUMMARY

In general terms, the present disclosure is directed to tracking catheter movement. In one possible configuration, a catheter tracking system obtains movement data from one or more encoders, analyzes the encoder data, and determines catheter movement data. Example catheter movement data include positional data, speed data, and directional data. Various aspects are described in this disclosure which include, but are not limited to, the following aspects.

One aspect is a catheter tracking system. In this aspect, the catheter tracking system includes a roller arranged to guide delivery of a catheter, an encoder arranged to obtain roller motion data, a processing unit, and memory. The memory stores instructions that, when executed by the processing unit, cause the catheter tracking system to acquire roller motion data and, using the roller motion data, determine catheter motion data. The catheter motion data includes at least one of: a catheter speed, a catheter direction, and a catheter position.

Another aspect is a method for determining motion data and position data of a catheter. In this aspect, the method includes receiving the catheter in a roller assembly, the roller assembly including a first roller and a second roller; obtaining first roller motion data; obtaining second roller motion data; and, using the first roller motion data and the second roller motion data, determining catheter motion data. The catheter motion data includes at least one of: catheter speed, a catheter direction, and a catheter position.

Yet another aspect is a catheter tracking system. In this aspect, the catheter tracking system includes a roller arranged to guide delivery of an imaging catheter, the roller including a first roller and a second roller; an encoder arranged to obtain roller motion data; a processing unit; and memory. The memory stores instructions that, when executed by the processing unit, cause the catheter tracking system to: obtain the roller motion data; using the roller motion data, determine catheter motion data, wherein the catheter motion data includes: a catheter speed, a catheter direction, and a catheter position; and transmit the catheter motion data to a display unit.

### BRIEF DESCRIPTION OF THE DRAWINGS

FIG. 1 is a sectional view of a prior art hemostasis valve.

FIG. 2 is a schematic illustration of an example catheter tracking environment.

FIG. 3 is a partial side view of a hemostasis valve and an embodiment of a catheter tracking system used in the environment of FIG. 2.

FIG. 4 is a partial side view of the hemostasis valve and catheter tracking system of FIG. 3 in a different operational position.

FIG. 5 is a partial end view of the hemostasis valve and catheter tracking system of FIG. 3.

FIG. 6 is a partial top view of the hemostasis valve and catheter tracking system of FIG. 3.

FIG. 7 is a sectional view of an embodiment of a catheter tracking system used in the environment of FIG. 2.

FIG. 8 is schematic diagram of catheter tracking system computing components in the embodiment shown in FIG. 7.

FIG. 9 is a sectional view of another embodiment of a catheter tracking system used in the environment of FIG. 2.

FIG. 10 is schematic diagram of catheter tracking system computing components in the embodiment shown in FIG. 9.

FIG. 11 is a sectional view of another embodiment of a catheter tracking system used in the environment of FIG. 2.

FIG. 12 is schematic diagram of catheter tracking system computing components in the embodiment shown in FIG. 11.

FIG. 13 is a partial side view of a hemostasis valve and an embodiment of a catheter tracking system.

FIG. 14 is schematic diagram of catheter tracking system electronic components used in the embodiment shown in FIG. 11.

FIG. 15 is a partial top view of a hemostasis valve, catheter tracking system, and catheter in accordance with an embodiment.

FIG. 16 is an example method for determining motion data and position data of a catheter using the example system of FIG. 13.

### DETAILED DESCRIPTION

Various embodiments will be described in detail with reference to the drawings, wherein like reference numerals represent like parts and assemblies throughout the several views.

Generally, the present disclosure is directed to catheter tracking. More particularly, the present disclosure includes systems and methods related to tracking catheter movement and position during medical procedures. Typically, systems configured for tracking catheter movement include a catheter tracking device. In some instances, systems can also include a hemostasis valve that can be used for catheter delivery during cardiovascular interventions. Of course, it will be appreciated that the systems and methods disclosed herein are not limited to tracking catheters during cardiovascular interventions.

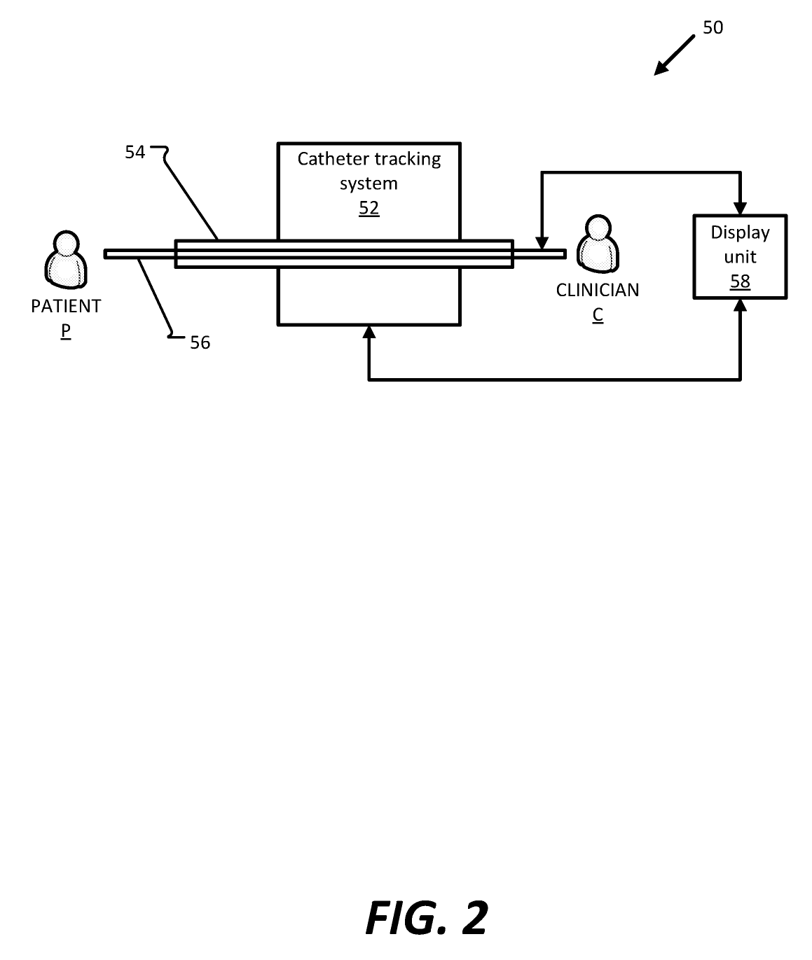

FIG. 2 is a schematic illustration of example catheter tracking environment 50. Example catheter tracking environment 50 includes catheter tracking system 52, valve 54, catheter 56, and display unit 58. Typically, catheter tracking environment 50 is a medical environment including clinician C and patient P. Other embodiments can include more or fewer components.

Clinician C interacts with various components of example catheter tracking environment 50. For example, clinician C can manipulate catheter 56 during a medical procedure involving patient P. During the medical procedure, clinician C can view data on one or more devices, such as display unit 58. Example data includes catheter position data, catheter speed data, and catheter direction data. In some implementations, clinician C can view image data from catheter 56 on display unit 58. In some implementations, clinician C can view signal data from catheter 56 on display unit 58. An example of signal data is pressure data, as discussed in further detail herein. Clinician C is usually a trained medical professional.

Catheter tracking system 52 determines movement and/or position data of catheter 56. In turn, catheter tracking system 52 can transmit movement and/or position data to display unit 58 for viewing by clinician C. Communication between catheter tracking system 52 and external devices, such as display unit 58, can be via wired or wireless connections. Catheter tracking system 52 can store movement and/or position data locally and/or remotely.

In an exemplary embodiment, catheter tracking system 52 provides catheter position tracking while also having a small form factor that is light weight. Catheter tracking system 52 can also be capable of wireless transmission. In an exemplary embodiment, use of catheter tracking system 52 results in minimal disruption to standard clinical workflows. For instance, in some implementations, use of catheter tracking system 52 enables control of catheter position by clinician C and maintaining a sterile field without use of a sterile drape.

Catheter 56 can be an imaging catheter. Catheter 56 can transmit imaging data to display unit 58 via wired and/or wireless connections. In some instances, movement and/or position data can be linked to imaging data obtained by catheter 56. Thereby, particular frames, images, portions of video, etc., can be mapped to movement or position data.

Catheter 56 can be a pressure sensing catheter. In some instances, catheter 56 can provide pressure data from pressure measurements to display unit 58. In turn, pressure data can be displayed by display unit 58.

Display unit 58 receives and displays data from various components in example catheter tracking environment 50. For instance, display unit 58 displays movement data transmitted by catheter tracking system 52. As another example, display unit 58 displays image data transmitted by catheter 56. In some instances, display unit 58 is integral with catheter tracking system 52 and is not an external device.

Display unit 58 can include memory, one or more processing units, and one or more internal or external display monitors. Display unit 58 can receive and coordinate data received from catheter tracking system 52 and catheter 56, such as mapping image data to position data.

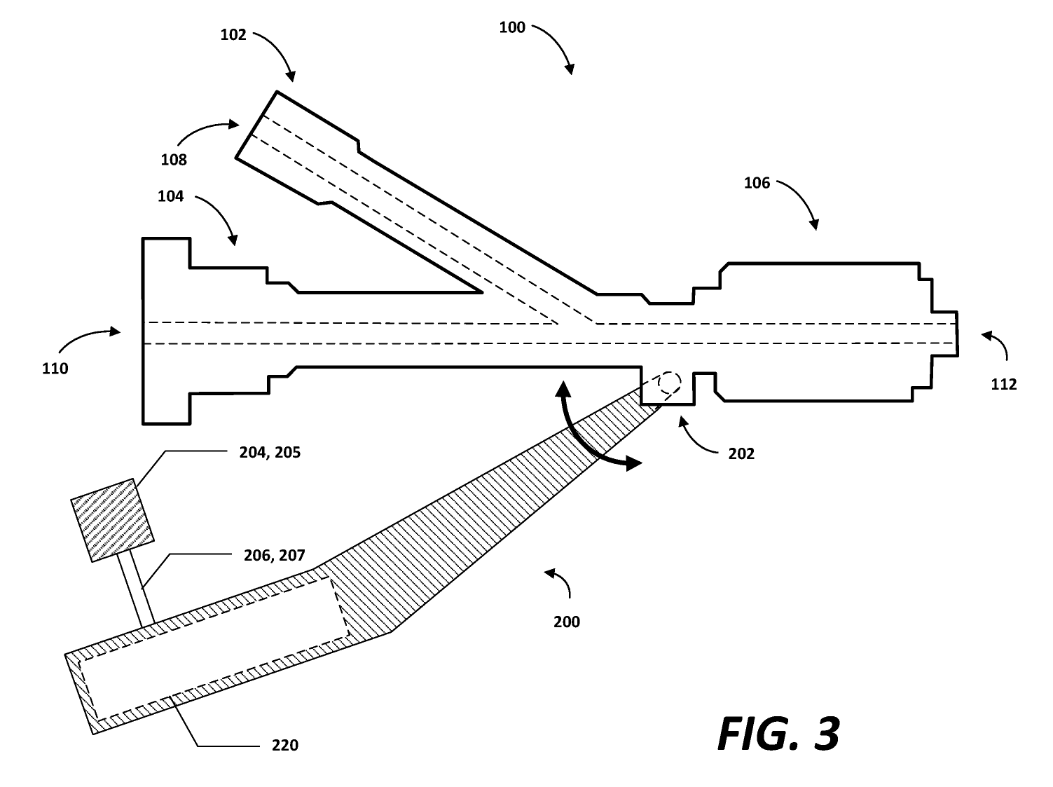

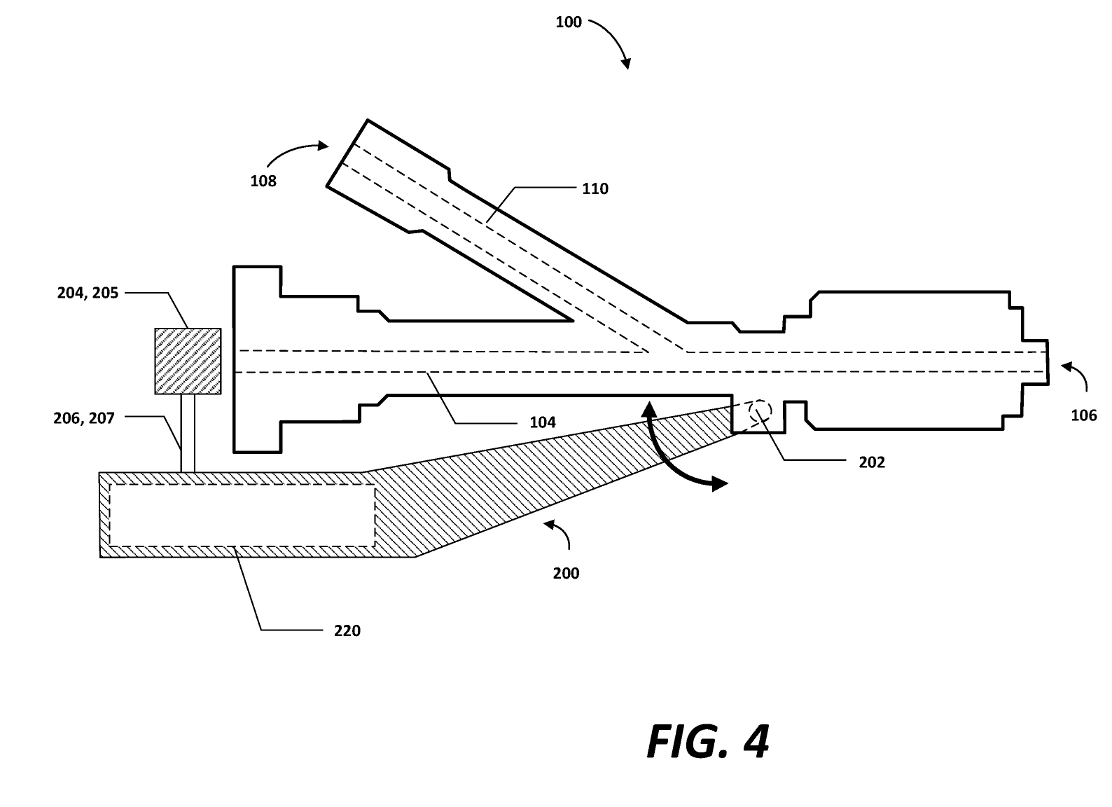

Referring to FIGS. 3 and 4, a partial side view of a hemostasis valve 100 and tracking device 200 according to one embodiment is shown. The hemostasis valve 100 includes a flush port 102, a valve 104, and a luer fitting 106. A guiding catheter (not shown) that is positioned within a patient's vascular system may be connected to the luer fitting 106. Saline or other fluids such as radiopaque contrast media may be delivered through the flush port lumen 108 to the guiding catheter. An intravascular device, such as an intracoronary imaging catheter, can be delivered through an entry port 110, an exit port 112, and into guiding catheter in the vascular system of a patient. The valve 104 may have a Tuohy-Borst valve design that includes a seal to minimize blood loss during a diagnostic or interventional procedure. The hemostasis valve 100 is generally composed of a biocompatible polymer, such as polycarbonate, and may accept catheters up to 9 Fr (3 mm outer diameter) in size, typically 7 Fr or smaller.

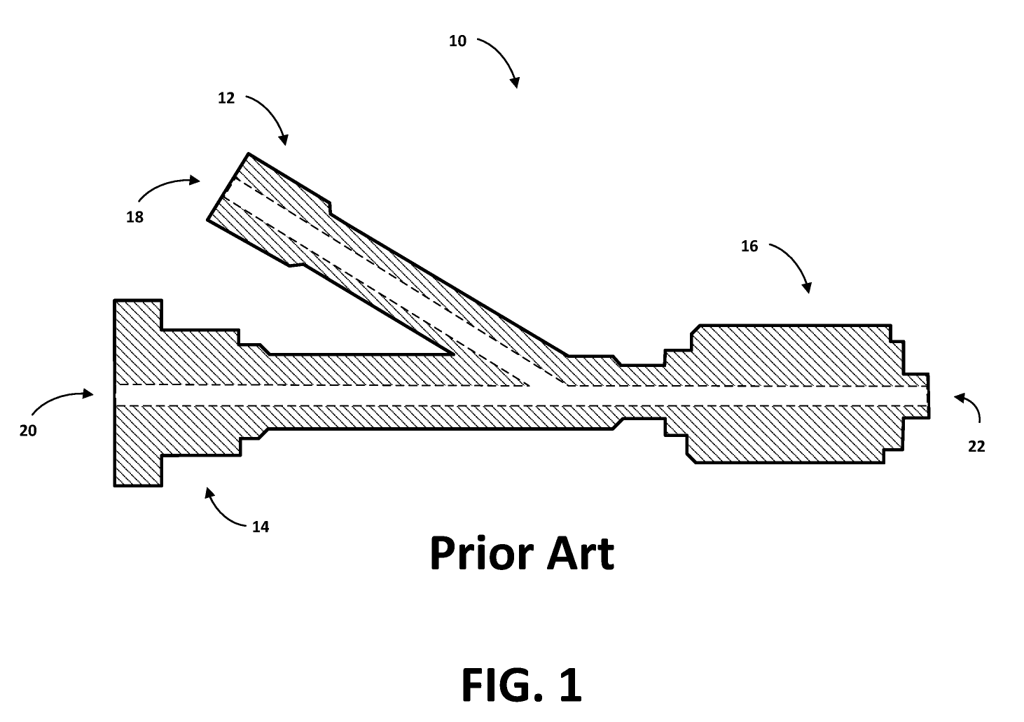

The tracking device 200 includes a hinge member 202, first and second guide rollers 204, 205, first and second guide roller shafts 206, 207, and a tracking device electronics assembly 220. The hinge member 202 enables an operator to position the tracking device 200 in an off position (e.g., positioned down as shown in FIG. 2) or an on position (e.g., positioned up as shown in FIG. 4). The hemostasis valve 100 and tracking device 200 may each include a magnet (not shown) of opposite polarity to facilitate locking of the tracking device 200 into the on position. When the tracking device 200 is in the off position an operator can use the hemostasis valve 100 in a similar manner as the prior art hemostasis valve 10 shown in FIG. 1.

The guide rollers 204, 205 are made of a biocompatible polymer, such as polyurethane. The guide rollers 204, 205 may have a diameter between 2 mm and 10 mm, typically approximately 6 mm. A 6.35 mm (¼″) diameter guide roller has a circumference of approximately 20 mm. The guide rollers shafts 206, 207 are generally rigid and made of a biocompatible material, such as stainless steel. The diameter of the guide roller shafts 206, 207 may be between 1 mm and 6 mm, typically 2 mm.

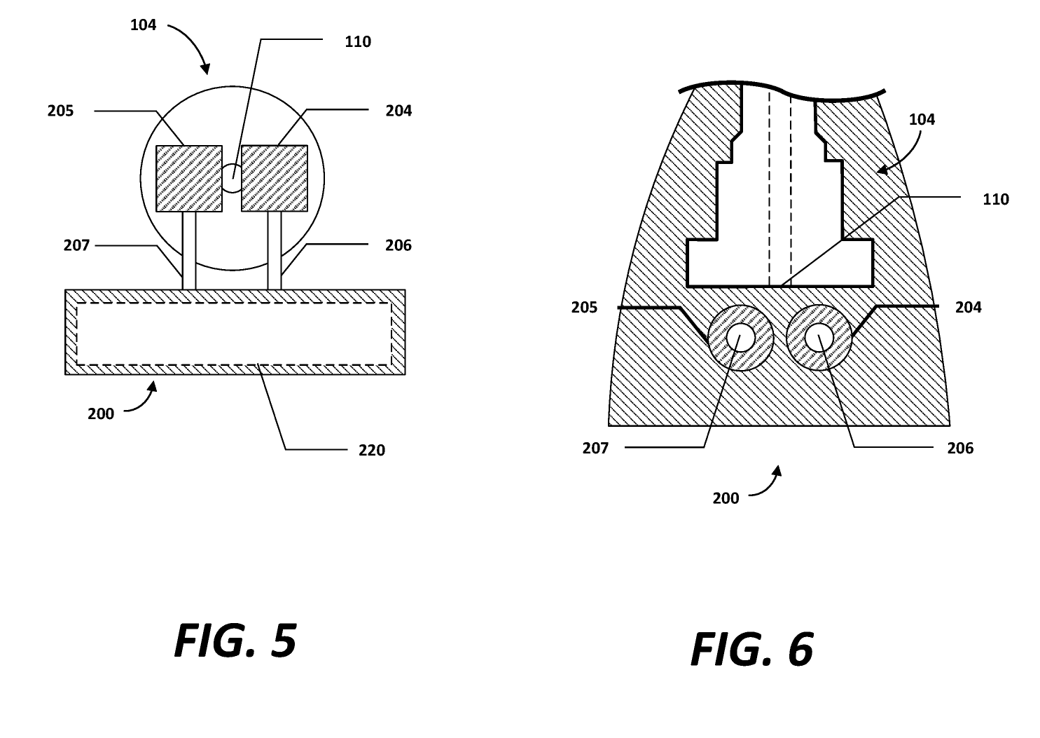

Referring now to FIGS. 5 and 6, a partial end view and a partial top view, respectively, illustrate the relative positions of the valve 104, entry port 110, and guide rollers 204, 205. An operator may deliver a catheter between the guide rollers 204, 205 and through the entry port 110.

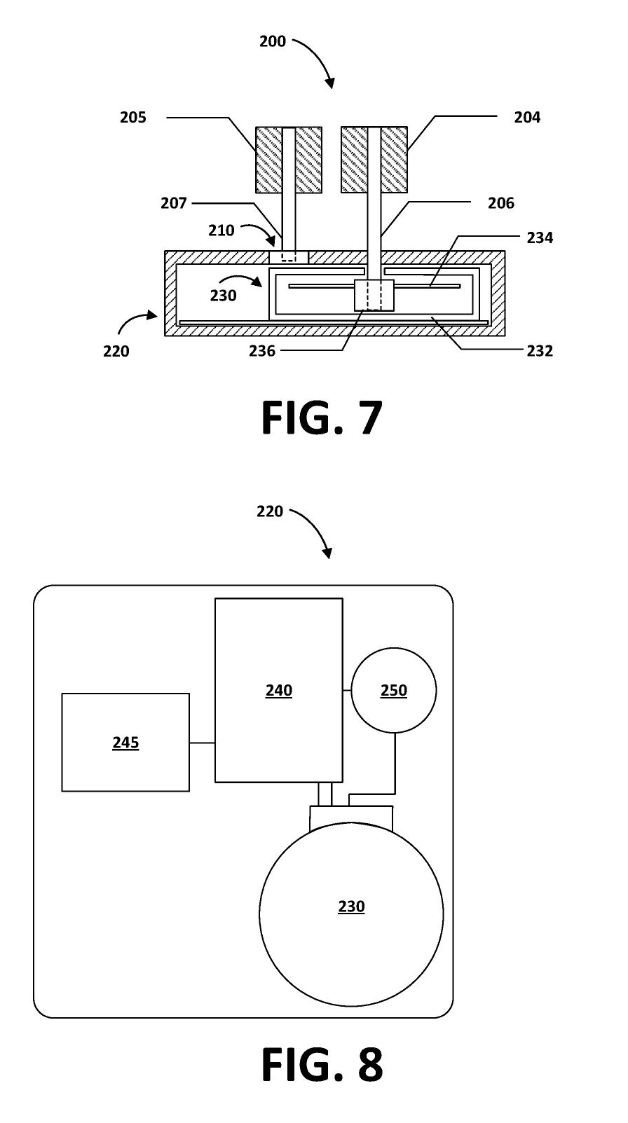

Referring now to FIG. 7, a sectional view of the tracking device 200 according to one embodiment is shown. The tracking device electronics assembly 220 includes a rotary optical encoder 230 that is coupled to the guide roller 204 and guide roller shaft 206. The rotary optical encoder 230 includes a rotary optical encoder housing 232, a rotary encoder disk 234, and a hub 236. Rotational motion of the guide roller 204 can be tracked by the rotary optical encoder 230 wherein a light source (not shown) and light detector (not shown) enable detection of rotation of the rotary encoder disk 234. The guide roller shaft 207 is coupled to a hub 210 that enables low-friction rotation of the guide roller 205.

Referring now to FIG. 8, a schematic diagram of the tracking device electronics assembly according to one embodiment is shown. The tracking device electronics assembly 220 includes the rotary optical encoder 230, an integrated microprocessor and wireless transmitter 240, memory 245, and a battery power system 250. The rotary optical encoder 230 may be a single-ended electrical design that includes connections for a supply voltage, a ground, a first quadrature signal, and a second quadrature signal. The required supply voltage may be between 4.5 V and 5.5 V, typically 5 V, and provided by the battery power system 250.

The first and second quadrature signals are transmitted from the rotary optical encoder 230 to the integrated microprocessor and wireless transmitter 240. The battery power system 250 provides an input/output supply voltage for the integrated microprocessor (e.g., ARM Cortex microcontroller) and wireless transmitter 240 that may be between 2.7 V and 3.6 V, typically 3.3 V. The battery power system 250 may further provide a battery supply voltage for the integrated microprocessor and wireless transmitter 240 that may be between 3.0 V and 4.3 V, typically 3.6 V.

The integrated microprocessor and wireless transmitter 240 receives the first and second quadrature signals from the rotary optical encoder 230 and may further process the quadrature signals before wirelessly transmitting information to an external device (not shown), such as a catheter-based imaging system. The rotary optical encoder 230 can track between 32 and 5000 positions of the guide roller 204. In an exemplary embodiment, a guide roller with diameter 6.35 mm and a rotary optical encoder with resolution of 2000 positions can track changes in catheter position as small as approximately 10 μm. The integrated microprocessor and wireless transmitter 240 may transmit the information by common wireless standards, such as Wi-Fi (e.g., IEEE 802.11) or Bluetooth.

Memory 245 stores one or more applications configured to perform one or more processes described herein. Memory 245 includes physical memory and/or computer readable storage media programmed according to the teachings of the present disclosure. Appropriate software coding can readily be prepared by skilled programmers based on the teachings of the present disclosure, as will be apparent to those skilled in the software art.

In some embodiments, the present disclosure includes a computer program product which is a non-transitory computer readable storage medium (media) having instructions stored thereon/in which can be used to program a computer to perform any of the processes of the present invention. Examples of storage mediums can include, but are not limited to, floppy disks, optical discs, DVD, CD-ROMs, microdrive, and magneto-optical disks, ROMs, RAMs, EPROMs, EEPROMs, DRAMs, VRAMs, flash memory devices, magnetic or optical cards, nanosystems (including molecular memory ICs), or other types of storage media or devices suitable for non-transitory storage of instructions and/or data.

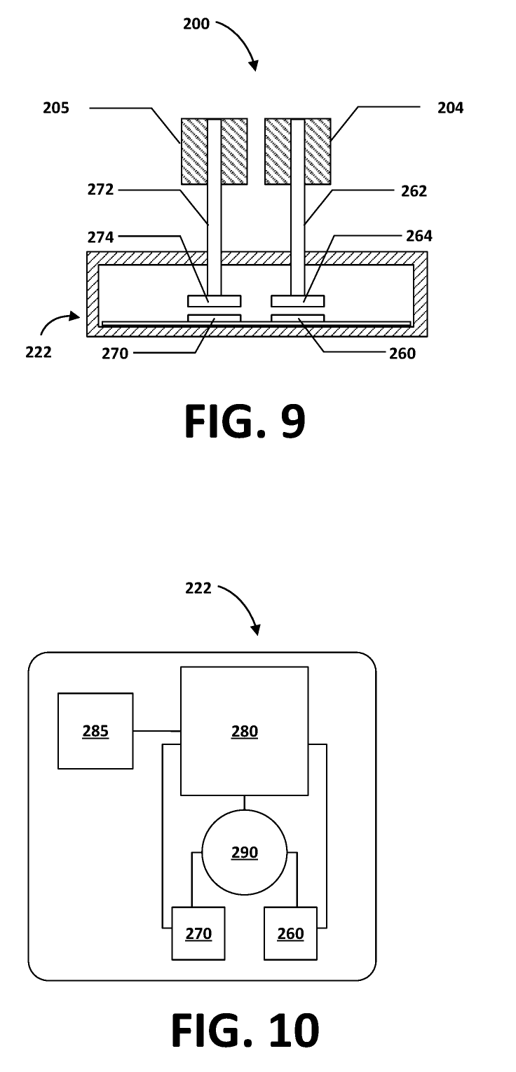

Referring now to FIGS. 9 and 10, another embodiment uses magnetic position sensing technology instead of optical technology to track device position and motion. The tracking device 200 includes first and second guide rollers 204, 205, a first guide roller shaft 262, a second guide roller shaft 272, a first magnet 264, a second magnet 274, and a tracking device electronics assembly 222. The first and second magnets 264, 274 may be neodymium disc magnets that are diametrically magnetized and be between 2 mm and 6 mm, generally 4 mm, in diameter. The first and second magnets 264, 274 are coupled respectively to the first and second guide roller shafts 262, 272.

The tracking device electronics assembly 222 includes a first magnetic position sensor 260, a second magnetic position 270, an integrated microprocessor and wireless transmitter 280, memory 285, and a battery power system 290. The first and second magnetic position sensors 260, 270 may each include at least one Hall sensor, typically four Hall sensors, to respectively detect the angle of the first and second magnets 264, 274. The first and second magnetic position sensors 260, 270 may each further include an analog-to-digital converter (ADC) to digitize an analog Hall effect sensor signal before further digital signal processing to calculate an angle of the first and second magnets 264, 274. The first and second magnetic position sensors 260, 270 transmit the angles of the first and second magnets 264, 274 to the integrated microprocessor and wireless transmitter 280. The magnetic position sensors 260, 270 can have resolutions between 8-bit (256 positions per revolution) and 14-bit (16,384 positions per revolution). In an exemplary embodiment, a 12-bit magnetic position sensor and a 6.35 mm diameter guide roller can track changes in catheter position as small as approximately 5 μm. The presence of two sensors, instead of only one, enables error checking to detect slippage between roller and catheter. The battery power system 290 may provide a supply voltage for the first and second magnetic position sensors 260, 270 that is between 2.7 V and 3.6 V, typically 3.3 V. The battery power system 290 may further provide a supply voltage for the integrated microprocessor and wireless transmitter 280 that is between 3.0 V and 4.3 V, typically 3.3 V.

The integrated microprocessor and wireless transmitter 280 may transmit the information by common wireless standards, such as Wi-Fi (e.g., IEEE 802.11) or Bluetooth. Memory 285 stores one or more applications configured to perform one or more processes described herein. Memory 285 includes physical memory and/or computer readable storage media programmed according to the teachings of the present disclosure. Appropriate software coding can readily be prepared by skilled programmers based on the teachings of the present disclosure, as will be apparent to those skilled in the software art.

In some embodiments, the present disclosure includes a computer program product which is a non-transitory computer readable storage medium (media) having instructions stored thereon/in which can be used to program a computer to perform any of the processes of the present invention. Examples of storage mediums can include, but are not limited to, floppy disks, optical discs, DVD, CD-ROMs, microdrive, and magneto-optical disks, ROMs, RAMs, EPROMs, EEPROMs, DRAMs, VRAMs, flash memory devices, magnetic or optical cards, nanosystems (including molecular memory ICs), or other types of storage media or devices suitable for non-transitory storage of instructions and/or data.

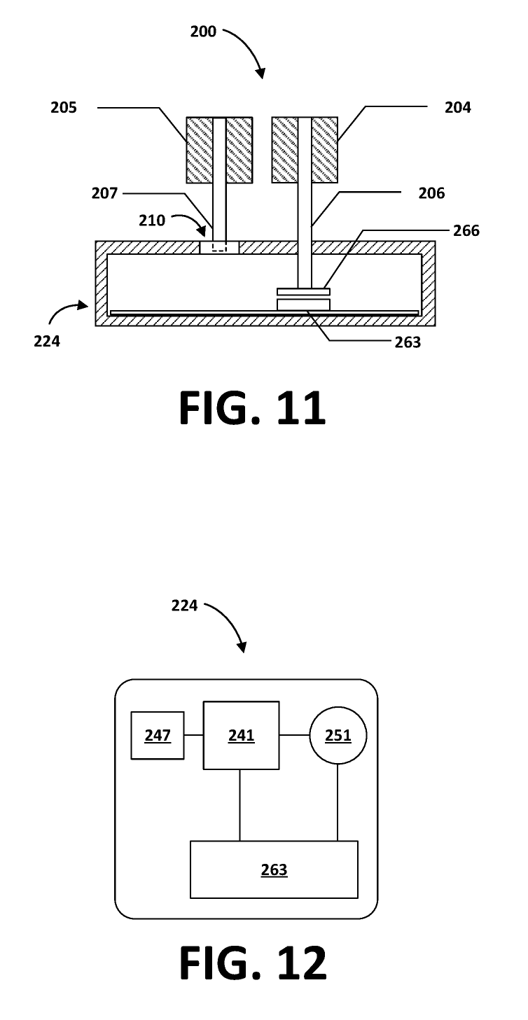

Referring now to FIGS. 11 and 12, still another embodiment uses optical navigation technology to track device position and motion. In some embodiments the optical navigation technology is similar to technology found in optical mouse devices. Rotational motion of the guide roller 204 can be tracked by an optical sensor 263 that enables detection of motion of a textured disk 266 wherein the textured disk 266 has a textured surface. In some embodiments the optical sensor 263 includes a light source (such as a light-emitting diode (LED) array), a lens, and an image acquisition system that acquires microscopic surface images of the textured disk.

The tracking device electronics assembly 224 includes the optical sensor 263, an integrated microprocessor and wireless transmitter 241, memory 247, and a battery power system 251. The optical sensor 263 detects the relative motion of the textured disk 266. The optical sensor 263 acquires sequential surface images of the textured disk 266 and determines the direction and magnitude of movement. The direction and magnitude of movement of the textured disk are sent to the integrated microprocessor and wireless transmitter 241. Memory 247, having similar components and functionality to memory 245 and memory 285 discussed above, stores applications enabling functionalities described herein.

In an exemplary embodiment, the optical sensor 266 has a resolution of 800 counts per inch (or approximately 30 μm) and can track motion up to 14 inches per second (or approximately 35 cm/s). The battery power system 251 may provide a supply voltage for the optical sensor 263 that is between 4.25 V and 5.5 V, typically 5.0 V. The battery power system 290 may further provide a supply voltage for the integrated microprocessor and wireless transmitter 241 that is between 3.0 V and 4.3 V, typically 3.3 V.

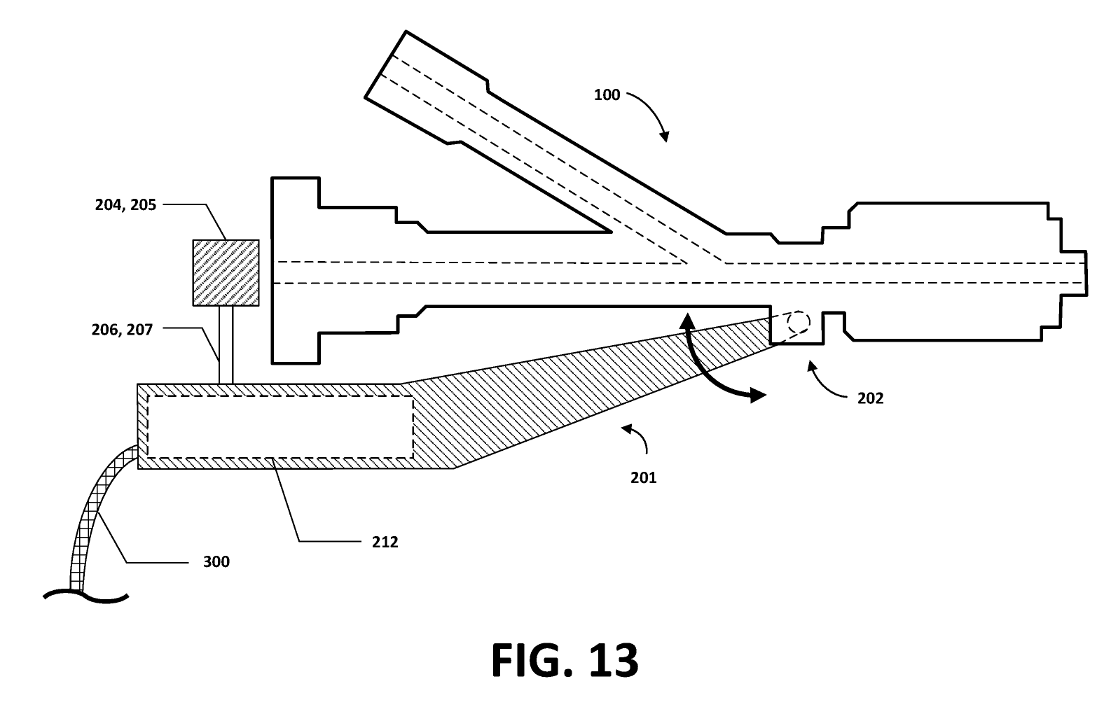

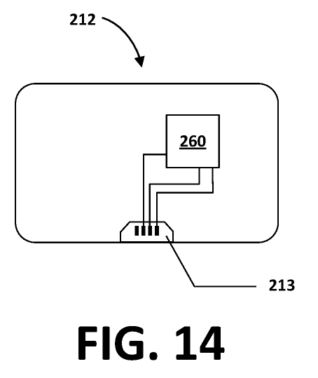

Referring now to FIGS. 13 and 14, still another embodiment includes an electrically wired design instead of a battery powered and wireless design. A tracking device 201 includes the hinge member 202, first and second guide rollers 204, 205, first and second guide roller shafts 206, 207, a tracking device electronics assembly 212, and a cable 300. The tracking device electronics assembly 212 includes the magnetic position sensor 260 and an electrical connector 213. The electrical connector 213 can be used to connect to an external medical device, such as a catheter-based imaging system, that provides at least a supply voltage and transfers signals for the angle of a magnet of the magnetic position sensor 260.

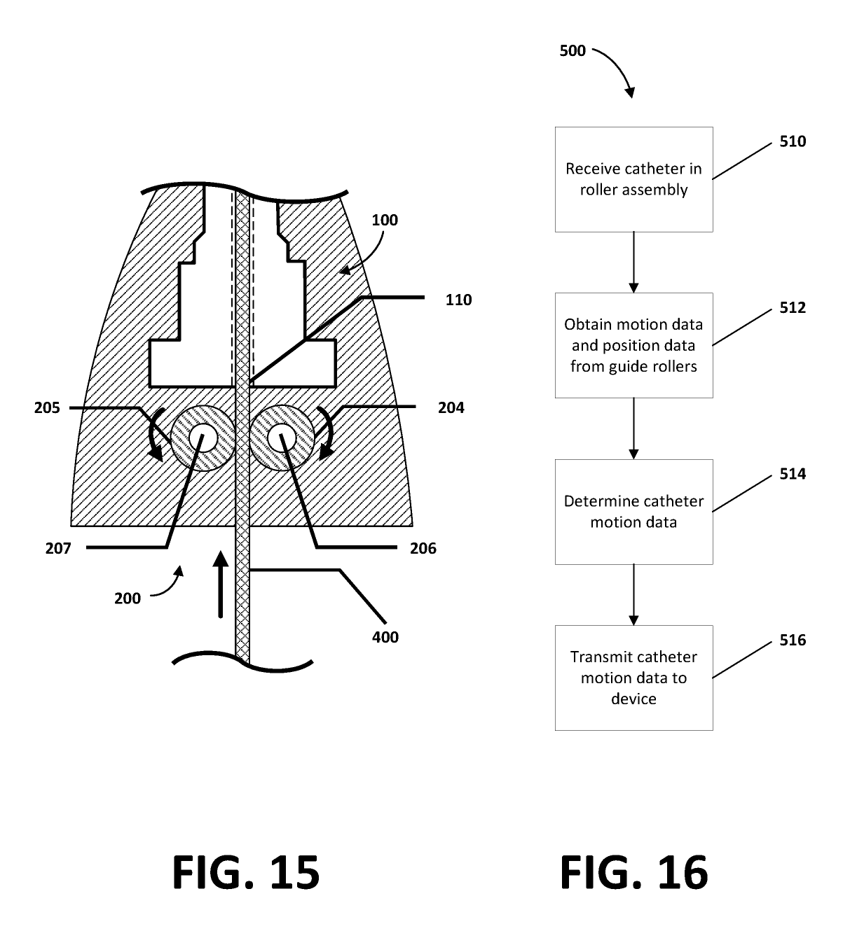

FIG. 16 shows example method 500 for tracking motion and position of a catheter. FIG. 15 is a schematic illustration of a portion of a catheter tracking system, and includes catheter 400 being delivered through a roller assembly that includes first guide roller 204 and second guide roller 205. FIGS. 15 and 16 are discussed concurrently below.

Example method 500 begins by receiving the catheter 400 through the roller assembly (operation 510), where the roller assembly includes first guide roller 204 and second guide roller 205. An operator, typically a clinician, delivers the catheter 400 through the roller assembly and into the entry port 110 of hemostasis valve 100. As the catheter 400 is delivered into the hemostasis valve 100, the first guide roller 204 rotates in a clockwise manner and the second guide roller 205 rotates in a counterclockwise manner.

As catheter 400 is delivered, motion data and position data from the roller assembly is obtained (operation 512). In some instances, obtaining motion data and position data (operation 512) includes receiving data relating to motion and position of one of the rollers 204 or 205. In other instances, obtaining motion data and position data (operation 512) includes receiving data relating to motion and position of both rollers 204 and 205.

Using the motion data and position data obtained from the roller assembly, catheter motion data are determined (operation 514). Catheter motion data can include a catheter speed, a catheter direction, and a catheter position. Then, catheter motion data are transmitted to a device (operation 516). Typically, the device is an external device that is capable of receiving and displaying catheter motion data. An example device is a display unit of a catheter-based imaging system.

What is claimed is:

1. A catheter tracking system, comprising:
a roller arranged to guide delivery of a catheter;
an encoder arranged to acquire roller motion data;
a processing unit; and
memory storing instructions that, when executed by the processing unit, cause the catheter tracking system to:
obtain the roller motion data; and
using the roller motion data, determine catheter motion data, wherein the catheter motion data includes at least one of: a catheter speed, a catheter direction, and a catheter position.

2. The catheter tracking system according to claim 1, wherein the catheter is an imaging catheter or a pressure sensing catheter.

3. The catheter tracking system according to claim 2, wherein the catheter is an intracoronary imaging catheter.

4. The catheter tracking system according to claim 1, wherein the memory further stores instructions that, when executed by the processing unit, cause the catheter tracking system to transmit the catheter motion data to a display unit.

5. The catheter tracking system according to claim 1, wherein the catheter motion data includes at least two of: the catheter speed, the catheter direction, and the catheter position.

6. The catheter tracking system according to claim 5, wherein the catheter motion data includes the catheter speed, the catheter direction, and the catheter position.

7. The catheter tracking system according to claim 1, wherein the catheter tracking system is coupled to a hemostasis valve.

8. The catheter tracking system according to claim 1, wherein the encoder is a rotary optical encoder.

9. The catheter tracking system according to claim 8, the rotary optical encoder including a rotary optical encoder housing, a rotary encoder disk, and a hub.

10. The catheter tracking system according to claim 1, wherein the encoder is a magnetic position sensor;
wherein the magnetic position sensor includes a first magnet and a second magnet; and
wherein the magnetic position sensor is configured to acquire first magnet angle data and second magnet angle data.

11. The catheter tracking system according to claim 1, wherein the encoder is an optical sensor, the optical sensor including a light source, a lens, and an image acquisition system.

12. The catheter tracking system according to claim 1, further comprising a hinge member, the hinge member being positionable in an off position and in an on position.

13. A method for determining motion data and position data of a catheter, the method comprising:
receiving the catheter in a roller assembly, the roller assembly including a first roller and a second roller;
obtaining first roller motion data;
obtaining second roller motion data; and
using the first roller motion data and the second roller motion data, determining catheter motion data, wherein the catheter motion data includes at least one of: catheter speed, a catheter direction, and a catheter position.

14. The method according to claim 13, further comprising transmitting the catheter motion data to a display unit.

15. The method according to claim 13, further comprising receiving image data from the catheter.

16. The method according to claim 15, further comprising mapping the catheter motion data to the image data.

17. A catheter tracking system, comprising:
a roller arranged to guide delivery of an imaging catheter, the roller including a first roller and a second roller;
an encoder arranged to obtain roller motion data;
a processing unit; and
memory storing instructions that, when executed by the processing unit, cause the catheter tracking system to:
obtain the roller motion data;
using the roller motion data, determine catheter motion data, wherein the catheter motion data includes: catheter speed data, catheter direction data, and catheter position data; and
transmit the catheter motion data to a display unit.

18. The catheter tracking system according to claim 17, further comprising a hinge member, the hinge member being positionable in an off position and in an on position, and
wherein the catheter tracking system is coupled to a hemostasis valve.

19. The catheter tracking system according to claim 18, wherein the encoder is a rotary optical encoder including a rotary optical encoder housing, a rotary encoder disk, and a hub.

20. The catheter tracking system according to claim 18, wherein the encoder is a magnetic position sensor including a first magnet and a second magnet; and
wherein the magnetic position sensor is configured to acquire first magnet angle data and second magnet angle data.
